(19) United States
(12) Patent Application Publication
(10) Pub. No.: US 2015/0125409 A1
(43) Pub. Date: May 7, 2015

(54) Ultrasonic Sunless Spray Tanning Device And Method

(71) Applicant: NORVELL SKIN SOLUTIONS, LLC, Alexandria, TN (US)

(72) Inventors: Nathan D. Marling, Lebanon, TN (US); Jason B. Selby, Lebanon, TN (US)

(73) Assignee: NORVELL SKIN SOLUTIONS, LLC, Alexandria, TN (US)

(21) Appl. No.: 14/532,237

(22) Filed: Nov. 4, 2014

Related U.S. Application Data
(60) Provisional application No. 61/900,107, filed on Nov. 5, 2013.

(51) Int. Cl. A61M 35/00; A61K 8/04; A61Q 19/04; B05B 17/06

(52) U.S. Cl. CPC A61M 35/00; B05B 17/0623; B05B 17/0676; A61K 8/04; A61Q 19/04; A61K 2800/20
USPC 424/59; 239/102.2

(57) ABSTRACT

Disclosed are an ultrasonic sunless spray tanning device and method involving components for ejecting a tanning solution mist atomized by ultrasonic vibrations.

### TECHNICAL FIELD

The invention relates generally to human tanning devices and methods. More specifically, the invention is directed to an ultrasonic sunless spray tanning device and method.

### BACKGROUND OF THE INVENTION

Some spray tanning devices experience inconsistent spray uniformity and inadequate atomization prior to dispersal from the nozzle. Spray tanners are also prone to clogging within the spray nozzles because sunless spray tanning solution has a propensity to clog the small holes within the nozzles. In addition, conventional spray tanning devices cause overspray during application. Accordingly, there is a need in the art for an ultrasonic sunless spray tanning device and method that alleviate these situations.

### SUMMARY OF THE INVENTION

An ultrasonic sunless spray tanning device and method includes a high frequency generator, peristaltic pumps, solenoid valves, one or more solution containers, an air compressor, and an ultrasonic nozzle. The ultrasonic nozzle includes one or more piezoelectric transducers and an amplifying horn contained within the nozzle housing. In certain embodiments, the device further includes an enclosure and a support arm, or carriage, which houses the ultrasonic nozzle and other hardware.

### BRIEF DESCRIPTION OF THE DRAWINGS

FIG. 1 is a perspective view of an embodiment of an ultrasonic sunless spray tanning device according to the invention.

FIG. 2 is an exploded view of an embodiment of the ultrasonic nozzle according to the invention.

### DETAILED DESCRIPTION OF THE INVENTION

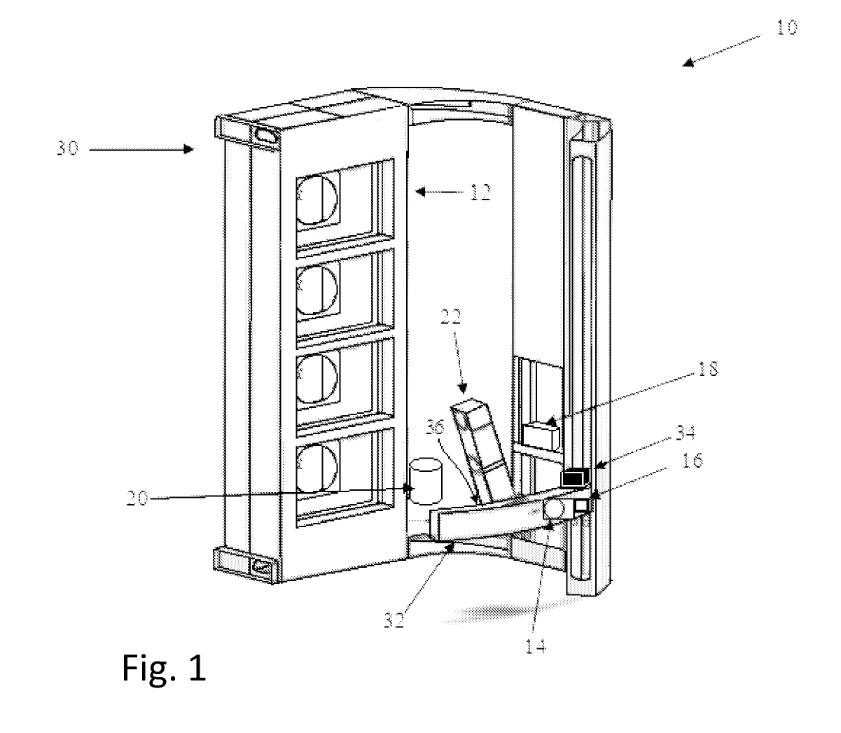

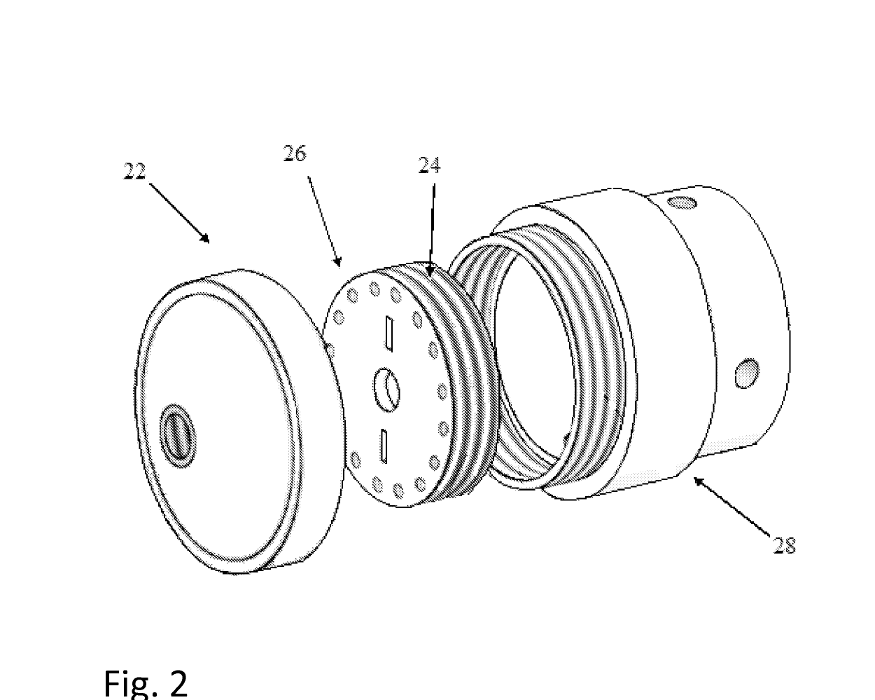

The invention will now be described with reference to the drawings. As shown in FIG. 1, an embodiment of the ultrasonic sunless spray tanning device 10 includes a high frequency generator 12, peristaltic pumps 14, solenoid valves 16, solution containers 18, an air compressor 20, and an ultrasonic nozzle 22. As shown in FIG. 2, the ultrasonic nozzle 22 includes one or more piezoelectric transducers 24 and an amplifying horn 26 contained within the nozzle housing 28. Embodiments of the device 10 may also include an enclosure 30 and a support arm, or carriage 32, which may house the ultrasonic nozzle 22 and other hardware. In other embodiments, the device 10 may include additional parts to impart portable spraying capabilities.

Ultrasonic Nozzle

The ultrasonic nozzle 22 has one or more piezoelectric transducers 24, which are driven by the high frequency generator 12. The ultrasonic nozzle 22 can be manufactured from any of several suitable materials, such as, but not limited to, stainless steel or any other material that exemplifies the conductive properties of stainless steel. In certain embodiments, the one or more piezoelectric transducers 24 are affixed to the amplifying horn 26, which can be manufactured from any of several suitable materials, such as, but not limited to, titanium or any other material that exemplifies the conductive properties of titanium. In use, a solution passes through a small orifice in the horn 26, where high frequency vibrations break down the solution into small particles, forming a mist. The pattern of the mist is dependent on the shape of the horn 26, which may vary depending on the specific application. Air pressure from the air compressor 20 is added to the nozzle 22, which adds force to eject the atomized solution from the nozzle 22. Optionally, the air or the solution may be heated to provide a more comfortable application.

The high frequency generator 12 provides a power source for the piezoelectric transducer 24. When power is supplied to the transducer 24, the transducer 24 produces a high frequency output. The output is directed at the amplifying horn 26, which amplifies the vibrations to the passing solution. The solution passes through a small orifice in the horn 26, where the high frequency vibrations break the solution into small particles, forming a fine mist. Air from the air compressor 20 enters through an opening in the nozzle 22, providing force to propel the atomized mist from the nozzle 22.

High Frequency Generator

The high frequency generator 12 produces a high frequency output, which is typically a sine wave frequency within the range of from about 20 to about 100 kHz. However, other frequencies are possible. The high frequency output provides electrical power that drives the piezoelectric transducers 24. The piezoelectric transducers 24 convert the output into vibrations. The piezoelectric transducers 24 are formed of a polarized piezoelectric ceramic, crystal, or other material suitable for the task. When an electrical oscillation is applied across the piezoelectric material, the polarized molecules align themselves with the electric field, resulting in dipoles within the molecular structure of the material. The piezoelectric material responds with a high frequency output, which acts as the ultrasonic source. The piezoelectric transducer 24 may be attached to the amplifying horn 13, which, in turn, amplifies the vibrations. The piezoelectric transducer 24 and the amplifying horn 13 are contained within the nozzle housing 28. As the solution passes through the amplifying horn 26, the high frequency vibrations break the solution into smaller particles.

Peristaltic Pumps

The device 10 further comprises peristaltic pumps 14 that carry the solution from the solution containers 18 to the ultrasonic nozzle 22, where the atomization occurs. Any suitable peristaltic pump 14 capable of pumping fluids may be used. A peristaltic pump 14 is generally defined as a pump that passes one or more roller surfaces over a compressible tube to force liquid through the tube. The rollers may be powered by a pump motor or any other suitable drive mechanism. In certain embodiments, the peristaltic pumps 14 contain at least four rollers to apply consistent compression to the tube. In certain embodiments, the peristaltic pumps 14 are bi-directional. Bi-directionally capable peristaltic pumps 14 allow for the capability of reversing direction of the liquid flow.

The liquids to be delivered via the peristaltic pumps 14 include a multitude of different solutions or compounds, ranging in viscosity from, for example, about 1 centipoise to in excess of about 15 centipoises. By way of non-limiting example, the device 10 can spray different shades of tanning solutions, prep sprays, pH balancers, fragrances, toners, post sprays, skin treatments, or other solutions or compositions.

Solenoid Valves

In certain embodiments, the device 10 further comprises solenoid valves 16 that control the flow of solution between the pumps 14 and the ultrasonic nozzle 22. The pumps 14 may operate with or without solenoid valves 16 to divert the flow of liquid. When present, each solenoid valve 16 may include an electronically-controlled three-way valve that can be opened or closed during the operation of the tanning process. Electrical switches control the opening or closing of the solenoid valves 16, thus changing the flow of the solution between the ultrasonic nozzle 22 and the solution container 18. In certain embodiments, the solenoid valves 16 are electronically controlled, and can be opened or closed electronically during operation. A metering valve may also establish pressure in the peristaltic pump 14 hoses. The solenoid valves 16 open and close to allow fluid to travel through hoses to reach the ultrasonic nozzle. Hoses may also allow excess solution to be returned to the solution container 18. The solenoid valves 16 may close to circulate the solution back into the solution container 18 or an alternative reservoir. In certain embodiments, a reverse action of the pumps 14 produces a return to the solution container 18 without the need of a separate hose or line. The solenoid valves 16 may be any suitable valves to perform these functions, or, alternatively, the direct actions of the peristaltic pumps 14 may perform these functions.

Solution Containers

Suitable solution containers 18 include any form of receptacle or reservoir to contain or deposit the liquid solutions. The solution container 18 deposits the liquid solutions for purposes of holding, storing, or transporting the solution to be atomized for spray applications. By way of non-limiting example, the solution containers 18 can be plastic containers or bags. The solution containers 18 may be situated internal or external to the device 10. In certain embodiments, the solution containers 18 are contained within a solution cabinet connected to the enclosure 30. In certain embodiments, a locking door is attached to the solution cabinet to prevent tampering. Multiple containers 18 may be implemented within the device 10 to facilitate spraying different solutions without requiring a change of containers or purging the hoses. Multiple solution containers 18 can hold different solutions for use in spray sessions or serve to collect returned solutions from the peristaltic pumps 14. This enables the device 10 to deliver, for example, different shades of tanning solutions.

The multiple solution containers 18 with different solutions or compounds are housed within the unit or in a receptacle nearby. Having multiple solution containers 18 allows the user to select a particular solution for application. The ability to apply different solutions without the need for purging hoses or changing containers creates efficiency for the operator and convenience for the receiver of the tanning solution. Furthermore, having multiple solution containers 18 enables the atomization and dispersal of solutions of different viscosities.

Air Compressor

The air compressor 20 is used to add force to the atomized mist as it is ejected from the ultrasonic nozzle 22. The air compressor 20 uses compressed air to apply the force used to eject the solution from the ultrasonic nozzle 22. Any form of compressor capable of providing the appropriate airflow through the ultrasonic nozzle 22 may be used as the air compressor 20. Compressed air from the air compressor 20 distributes the atomized particles out of the ultrasonic nozzle 22 in a fine mist. In certain embodiments, the ejected atomized mist is focused toward the inside of the enclosure 30. The air compressor 20 combines a stream of compressed air to the atomized tanning solution at or near the ultrasonic nozzle 22 outlet. The compressed air provides force to eject the atomized solution from the ultrasonic nozzle 22. The resulting mist can be applied to the surface of the skin to generate a sunless tan. In certain embodiments, the compressed air is heated to provide the user with a more comfortable spray session.

Enclosure

The device 10 may be configured within a spray tanning booth that is generally a free-standing structure, forming a partial enclosure that may have a base, floor, and top. In certain embodiments, the ultrasonic nozzle 22 is mounted on a motor-driven assembly, such as in an enclosed spray booth. Spray tanning devices may take many forms, and booths may be automated or manual. Non-limiting examples of spray tanning booths include, but are not limited to, the Norvell® Sunless Colosseum, the Norvell® Auto Revolution, and the Norvell® Auto Revolution Plus booths. In other embodiments, the ultrasonic nozzle 22 is mounted on a stationary or mobile platform. The ultrasonic nozzle 22 can be mounted on many other types of fixtures.

In an embodiment, the enclosure 30 is dimensioned so that a person may stand within it. The enclosure 30 assists in containing any overspray from the solution application process. Fans or turbines may be incorporated into the enclosure 30 to further prevent solution particles from circulating into the air or settling onto nearby surfaces. Fans act to provide suction for any overspray from the application process. The enclosure 30 may also be equipped with lighting, such as LEDs, to provide better illumination. Electric controls may be mounted onto the enclosure 30. These controls can be either manual, such as a key switch control, or digital, such as a touch screen interface.

In an embodiment, the enclosure 30 is a structural assembly with an opening and dimensions to satisfactorily allow a person to enter, stand, and maintain positions to better receive the atomized solution. The enclosure 30 acts as a housing for many of the device's components. Such components may include fans, ducting, pumps, solution containers, wiring, motors, and hoses.

The enclosure 30 may contain walls, a ceiling, and a floor, or the enclosure 30 may simply be a walled structure. The enclosure 30 may be mounted on wheels for easier movement. In instances where the mobility of the enclosure 30 could pose a hazard, wheels may be omitted in favor of a solid, free-standing structure. Several other variations exist for spray tanning booths, and are encompassed within the scope of the invention.

The enclosure 30 may further include elements of design, manufacture, or signage intended to educate the user on appropriate body positions for optimal application results. For example, users may be required to change standing orientation during the application process for optimal results.

The enclosure 30 may further contain a filter or filtration system, such a dual dry filter system, to further reduce breathable particles in the air. Heating pads or elements 34 may also be incorporated within the enclosure 30 for the dual purpose of providing a more comfortable experience and increasing the reactivity of, for example, dihydroxyacetone (DHA) on the skin. Higher temperature and increased surface coverage due to finely atomized particles produce better results from the sunless tanning application.

Connections of Elements

Tanning solution to be applied to skin is contained within the solution container 18. The solution is pumped through hoses using one or more peristaltic pumps 14. One or more solenoid valves 16 between the solution container 18 and ultrasonic nozzle 22 may control the route the solution travels. Electrical switches may control the opening or closing of the solenoid valves 16, thus changing the flow of the solution between the ultrasonic nozzle 22 and the solution container 18. The high frequency generator 12 provides electrical power for the piezoelectric transducer 24, which may be attached to the amplifying horn 26 within the nozzle housing 28. As the tanning solution passes through the amplifying horn 26, the high frequency vibrations atomize the liquid into a fine mist. Air from the air compressor 20 distributes the particles out of the ultrasonic nozzle 22 in a fine mist, focused toward the inside of the enclosure 30.

Operation

In embodiments featuring a booth or enclosure 30, the user enters the opening in the enclosure 30. The design, manufacture, and signage elements of the enclosure 30 provide guidelines for appropriate positions to hold the body in for best application results. The lights provide additional illumination for the user to see inside the enclosure 30. If present, the fans or turbines provide suction for any overspray from the application process. If present, the filter or filtration system further reduces breathable particles in the air. If present, the heating pads or elements 34 heat the air for the dual purpose of providing a more comfortable user experience and increasing the reactivity of, for example, DHA.

In certain embodiments, the ultrasonic nozzle 22 is mounted to an automated arm or stationary surface on the enclosure 30. By way of non-limiting example, the nozzle 22 may be fixed on a movable track. The automated arm, or carriage 32, may be bi-directionally moveable laterally along a carriage track 36 that traverses the enclosure opening. This movement is controlled by a carriage motor or carriage drive. In certain embodiments, the carriage 32 moves across an upper and lower carriage track 36. The ultrasonic nozzle 22 may be moveable longitudinally along a track mounted inside the carriage 32. The nozzle 22 is capable of projecting an atomized spray while moving, thus allowing the nozzle 22 to be repositioned while the user remains stationary.

In a typical use, the tanning solution is contained within the solution container 18 prior to application to the body. In some applications, hoses are attached to the peristaltic pumps 14 to provide a conduit for the solution to travel from the solution container 18 to the peristaltic pumps 14. Another set of hoses may connect to the peristaltic pumps 14 to act as a return line to the solution container 18 or an alternate solution container 18.

The solenoid valves 16 between the peristaltic pumps 14 and the ultrasonic nozzle 22 may be triggered electronically to allow the flow of solution to continue through hoses until it reaches an outlet at the ultrasonic nozzle 22. In some applications, the solenoid valves 16 may also be triggered electronically to direct the flow of sunless solution back into a solution container 18. In alternate embodiments, the actions of the solenoid valves 16 may be accomplished through the use of pumps 14.

The device 10 applies ultrasonic vibrations to an aqueous solution as it passes through the ultrasonic spray nozzle 22. This process begins with the high frequency generator 12, which creates a sine wave frequency which supplies power to the piezoelectric transducer 24. The piezoelectric transducer 24 is formed of a polarized piezoelectric ceramic, crystal, or other material suitable for the task. When an electrical oscillation is applied across the piezoelectric material, the polarized molecules align themselves with the electric field, resulting in dipoles within the molecular structure of the material. The piezoelectric material responds with a high frequency output, which acts as the ultrasonic source. The piezoelectric transducer 24 may be attached to the amplifying horn 26, but does not need to be, and the amplifying horn 26 amplifies the vibrations. As the solution passes through the amplifying horn 26, the high frequency vibrations break the solution into smaller particles, creating an atomized mist.

The air compressor 20 sends a stream of compressed air to the atomized solution at or near the ultrasonic nozzle 22 outlet. The air pressure provided by the air compressor 20 discharges the atomized solution from the ultrasonic nozzle 22 onto an external target such as skin. Atomization decreases the size of droplets expelled from the ultrasonic nozzle 22, which in turn reduces the amount of solution that attaches to the nozzle housing 28. In certain embodiments, the fine spray dispersed by the tanning device applies atomized solutions in an even distribution to the skin.

The solution may be heated prior to spraying in order to provide enhanced comfort. Accordingly, in certain embodiments, the device 10 further comprises a heater 34 configured to heat the solution prior to being sprayed. Alternatively, or in addition, the device 10 may comprise a heater 34 configured to heat the compressed air prior to ejecting the atomized mist through the ultrasonic nozzle 22.

In certain embodiments, the device 10 alleviates the problem of frequent clogging within the ultrasonic nozzle 22. Sunless tanning solution has a propensity to clog the small holes within current nozzles used in the art. The ultrasonic atomization decreases the size of droplets being expelled from the ultrasonic nozzle 22, thus reducing the amount of solution that attaches to the nozzle housing 28. In certain embodiments, the device 10 also disperses a fine spray of atomized solutions in an even distribution onto the skin. The device 10 provides the operator with greater control of the solution application and reduces the risk of over-spraying areas of the skin. In addition, the device 10 produces less overspray. Reducing overspray decreases both the amount of solution needed during application and the quantity of breathable particles in the air.

In an embodiment, the ultrasonic nozzle 22 is attached to an external device, such as a spray gun or existing mobile spray system, for use in a portable, handheld unit. A high viscosity, low pressure (HVLP) spray gun is a common instrument of spray tanning, but others are possible. By way of non-limiting example, the Norvell® VIP² HVLP Handheld System may be employed.

In other embodiments, the device 10 omits the solenoid valves 16 and incorporates other methods of controlling the flow of solution. By way of non-limiting example, pumps 14 may be located in close proximity to the ultrasonic nozzle 22 to allow activation of the pumps 14 to control the flow of liquids between the pumps 14 and the nozzle 22. Reversing the action of the pumps 14 allows the liquid solutions to be returned to the solution containers 18 without the need of solenoid valves 16.

Certain embodiments of the apparatus disclosed herein are defined in various examples. It should be understood that these examples, while indicating particular embodiments of the invention, are given by way of illustration only. From the above description and these examples, one skilled in the art can ascertain the essential characteristics of this disclosure, and without departing from the spirit and scope thereof, can make various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. An ultrasonic sunless spray tanning device comprising:
a container capable of containing a solution;
a nozzle having one or more transducers positioned adjacent to an amplifying horn;
a generator in communication with the one or more transducers, wherein the generator is capable of producing a high frequency output and the one or more transducers are configured to convert the high frequency output into ultrasonic vibrations;
a pump in fluid communication with the container and the nozzle, the pump being configured to carry the solution from the container to the nozzle, wherein the amplifying horn is configured to amplify the ultrasonic vibrations to the solution passing through the nozzle to create an atomized mist; and
a compressor configured to deliver pressurized fluid to the nozzle to eject the atomized mist.

2. The ultrasonic sunless spray tanning device of claim 1, comprising a plurality of containers, the device being capable of containing a plurality of solutions.

3. The ultrasonic sunless spray tanning device of claim 1, further comprising a heater configured to heat the solution or heat the pressurized fluid.

4. The ultrasonic sunless spray tanning device of claim 1, further comprising a solenoid valve configured to control the flow of solution between the pump and the nozzle.

5. The ultrasonic sunless spray tanning device of claim 4, wherein the solenoid valve comprises an electronically controlled three-way valve.

6. The ultrasonic sunless spray tanning device of claim 1, further comprising a metering valve to control pressure in the pump.

7. The ultrasonic sunless spray tanning device of claim 1, wherein the device includes an enclosure.

8. The ultrasonic sunless spray tanning device of claim 7, wherein the enclosure is a spray tanning booth.

9. The ultrasonic sunless spray tanning device of claim 8, wherein the nozzle is mounted on a carriage.

10. The ultrasonic sunless spray tanning device of claim 1, wherein the device is a spray gun.

11. An ultrasonic sunless spray tanning device comprising:
an enclosure housing a carriage and a carriage track; and
an ultrasonic spray nozzle mounted on the carriage, the ultrasonic spray nozzle comprising one or more piezoelectric transducers configured to create ultrasonic vibrations; and
an amplifying horn configured to amplify the ultrasonic vibrations;
wherein the carriage is bi-directionally moveable along the carriage track.

12. The ultrasonic sunless spray tanning device of claim 11, further comprising a carriage motor configured to control the movement of the carriage along the carriage track.

13. A method of applying a sunless tanning solution comprising:
transporting a sunless tanning solution from a solution container to an ultrasonic nozzle;
amplifying ultrasonic vibrations in the direction of the sunless tanning solution in the ultrasonic nozzle to atomize the sunless tanning solution into a mist; and
ejecting the mist from the ultrasonic nozzle.

14. The method of claim 13, wherein the ultrasonic nozzle comprises one or more piezoelectric transducers in communication with a generator capable of producing a high frequency output.

15. The method of claim 13, wherein an amplifying horn amplifies the ultrasonic vibrations.

16. The method of claim 13, wherein the ultrasonic nozzle is configured in a portable tanning spray gun.

17. The method of claim 13, wherein the ultrasonic nozzle is configured on a carriage in an enclosure.

18. The method of claim 13, wherein a solenoid valve controls the flow of solution from the solution container to the ultrasonic nozzle.

19. The method of claim 18, wherein the solenoid valve comprises an electronically controlled three-way valve.

20. The method of claim 13, wherein the solution is transported by a pump.
